(19) United States
(12) Patent Application Publication
(10) Pub. No.: US 2024/0298631 A1
(43) Pub. Date: Sep. 12, 2024

(54) SYSTEM AND METHOD FOR REAL-TIME MONITORING CONDITION OF ORGAN DURING TRANSPORT, TRANSPLANT, AND ARTIFICIALLY DIVERTED RECIRCULATORY PATHWAYS

(71) Applicant: RCE Technologies, Inc., Carlsbad, CA (US)

(72) Inventors: Atandra Burman, Bermuda Dunes, CA (US); Jitto Titus, Acworth, GA (US)

(73) Assignee: RCE Technologies, Inc., Carlsbad, CA (US)

(21) Appl. No.: 18/596,224

(22) Filed: Mar. 5, 2024

Related U.S. Application Data
(60) Provisional application No. 63/488,760, filed on Mar. 6, 2023.
(60) Provisional application No. 63/500,896, filed on May 8, 2023.
(60) Provisional application No. 63/488,750, filed on Mar. 6, 2023.

(51) Int. Cl. A01N 1/02; C12M 1/34

(52) U.S. Cl. CPC A01N 1/021; A01N 1/0247; A01N 1/0284; C12M 41/30

(57) ABSTRACT

Method or system for monitoring a condition of an organ, which may be a donor organ, using an optical device; providing a reservoir of normothermic fluid; providing a perfusion circuit for the normothermic fluid; and providing a pump in fluid communication with perfusion circuit. Providing an oxygenator for normothermic fluid during circulation through the perfusion circuit to perfuse the donor heart with oxygenated blood; an optical device integrated within the reservoir, or pump or oxygenator, or in-line flow-based within perfusion circuit through normothermic fluid flows. May use total internal reflection for optical device. Monitoring condition of an organ, circulation system for blood, exterior to a body, but connected to the body; and adding filtration to blood, and filtered blood goes back into body. Monitoring a condition of a heart or lung, using extracorporeal membrane oxygenation (ECMO) to monitor the oxygenation levels in circulation.

### CROSS-REFERENCE TO RELATED APPLICATIONS

This application claims the benefit of U.S. Provisional Application No. 63/488,760, filed 6 Mar. 2023; U.S. Provisional Application No. 63/500,896, filed 8 May 2023; and U.S. Provisional Application No. 63/488,750, filed 6 Mar. 2023. All of which are hereby incorporated by reference in their entirety.

### TECHNICAL FIELD

The present disclosure relates to a system, apparatus, and method using optical techniques for cardiac, or other organ, transplant monitoring.

### SUMMARY

A method or system for monitoring a condition of an organ, which may be a donor organ. The methods or systems may include using an optical device; providing a reservoir of normothermic fluid; providing a perfusion circuit for the normothermic fluid; and providing a pump in fluid communication with the perfusion circuit. The methods or systems may further include providing an oxygenator for oxygenating the normothermic fluid during circulation through the perfusion circuit to perfuse the donor heart with oxygenated blood; providing an optical device integrated within the reservoir, or the pump or the oxygenator, or in-line flow-based within the perfusion circuit through which the normothermic fluid flows; and using total internal reflection for the optical device. The methods or systems may further include that the reservoir, pump, oxygenator, or in-line perfusion circuit of normothermic fluid and the optical device are configured to monitor the physiological baseline of the donor organ during transport.

The methods or systems may further include monitoring a condition of an organ, including a circulation system for blood, exterior to a body, but connected to the body; and using the circulation system for adding filtration for the blood, and wherein the filtered blood goes back into the body. The methods or systems may further include an external circulation system integrates an optical device to monitor the physiological baseline of the heart.

The methods or systems may further include monitoring a condition of a heart or lung, using extracorporeal membrane oxygenation (ECMO) to monitor the oxygenation levels in circulation; and integrating an optical device to monitor the physiological baseline of the organ. The methods or systems may further include using total internal reflection for the optical device.

### BACKGROUND

While more than 100,000 patients could benefit from a new heart, less than 10% of donor hearts are transplanted into a patient in the United States each year. The ischemic time, or time out of the body for a donor heart, or other organ, is at most 4 to 6 hours. This does not leave much time to remove an organ, get it to the recipient, and complete a complicated transplant surgery. In some cases, the donor organ is a donor organ selected from a group consisting of a heart, a liver, a lung, or a kidney.

Improvement in monitoring the condition of a donor heart, or other transplant organ, during transport this could enable organ care systems (OCS) to evaluate a suboptimal heart they otherwise wouldn't risk using, for suitability of transplant, potentially increasing the percentage of donor hearts which can be transplanted.

### BRIEF DESCRIPTION OF THE DRAWINGS

The description herein makes reference to the accompanying drawings wherein like reference numerals refer to like parts throughout the several views.

FIG. 1 is a schematic illustration of an exemplary system for transporting a donor heart.

FIG. 2 is a schematic illustration of an example configuration of a system including an optical device integrated with in-line flow of normothermic fluid in the system of FIG. 1.

FIG. 3A is a schematic illustration of another example configuration of a top view of a system including an optical device integrated with in-line flow of normothermic fluid; and FIG. 3B is a schematic illustration of another example configuration of a top view of a system including an optical device integrated with in-line flow.

FIG. 4 is a schematic illustration of an example configuration of a system including an optical device integrated at multiple sites within the system of FIG. 1, for monitoring the condition of a donor heart during transport.

### DETAILED DESCRIPTION

In the following description, numerous details of the embodiments of the present disclosure the condition should be deemed merely as exemplary, are set forth with reference to accompanying drawings to provide thorough understanding of the embodiments of the present disclosure. Therefore, those skilled in the art will appreciate that various modifications and replacements may be made in the described embodiments without departing from the protection scope and the spirit of the present disclosure. Further, for clarity and conciseness, descriptions of known functions and structures are omitted hereinafter.

While more than 100,000 patients could benefit from a new heart, less than 10% of hearts are transplanted in the United States each year. The ischemic time, or time out of the body for a done heart, is at most 4 to 6 hours. This does not leave much time to remove an organ, get it to the recipient, and complete a complicated transplant surgery. Troponin, or Al-troponin, can monitor the tendency of donor hearts for transplants or transplant-ability. In some cases, the donor organ is a donor organ selected from a group consisting of a heart, a liver, a lung, or a kidney.

The methods also are configured to monitor inframarkers that include, without limitation, biomarkers of myonecrosis such as cardiac Troponin; biomarkers of myocardial ischemia such as heart type fatty acid binding protein, cytosolic cardiac Troponin and functional analogs of cardiac Troponin such as Tropomyosin and Troponin C; inflammatory biomarkers such as c-reactive protein, pentraxin3; biomarkers of liver function such as alanine aminotransferase (ALT), aspartate aminotransferase (AST), alkaline phosphatase (ALP), glutamyl transpeptidase (GGT), and total bilirubin (TBIL); biomarkers of acute kidney rejection such as MicroRNAs, dd-cf DNA, CD103 markers, CXCR3 chemokine receptor, IP-10, KIR genes, HLA antibodies, the perforin and granzyme B molecules; biomarkers of lung injury post lung transplant such as M30, M65, soluble receptor of advanced glycation end-products (sRAGE), and plasminogen activator inhibitor-1 (PAI-1), and inflammatory markers such as interleukins 6, 8, and 10 (IL-6, IL-8, IL-10) and tumor necrosis factor (TNF)-αAl-al-troponin.

While the present disclosure may be illustrated with respect to particular industries or applications, those skilled in the art will recognize the broader applicability of the products, methods, and techniques, described herein. For example, similar structures, methods, or combinations thereof, may be used in other industries or for other procedures/processes than those described herein. The order of any method or process steps described herein is not limiting.

Those having ordinary skill in the art will also recognize that terms such as “above,” “below,” “upward,” “downward,” et cetera, are used descriptively of the figures, and do not represent limitations on the scope of the appended claims. Any numerical designations, such as “first” or “second” are illustrative only and are not intended to limit the scope of the claims in any way.

When used herein, the term “substantially” refers to relationships that are ideally perfect or complete, but where manufacturing realities prevent absolute perfection. Therefore, substantially denotes typical variance from perfection in the relevant art. For example, if height A is substantially equal to height B, it may be preferred that the two heights are 100.0% equivalent, but manufacturing realities likely result in the distances varying from such perfection. Skilled artisans would recognize the amount of acceptable variance. For example, and without limitation, coverages, areas, or distances may generally be within 10% of perfection for substantial equivalence. Similarly, relative alignments, such as parallel or perpendicular, may generally be within 5%.

A generalized control system, computing system, or controller is operatively in communication with relevant components of, at least, the systems described herein. The controller includes, for example and without limitation, a non-generalized, electronic control device having a preprogrammed digital computer or processor, a memory, storage, or non-transitory computer-readable storage medium used to store data such as control logic, instructions, lookup tables, etc., and a plurality of input/output peripherals, ports, or communication protocols. One or more of the methods described herein may be executed by the controller, including the non-transitory computer-readable storage medium, or other structures or equipment recognizable to skilled artisans. For example, and without limitation, the AI methods may be applied to the systems described herein.

Features shown in one figure may be combined with, substituted for, or modified by, features shown in any of the figures. Unless stated otherwise, no features, elements, or limitations are mutually exclusive of any other features, elements, or limitations. Any specific configurations shown in the figures are illustrative only and the specific configurations shown are not limiting.

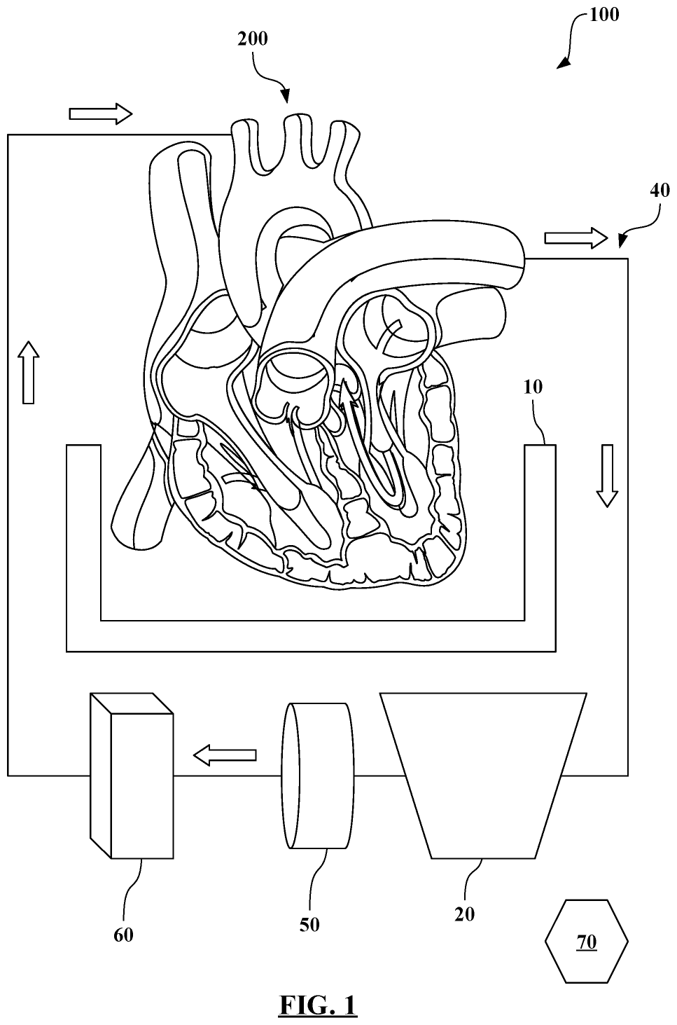

Referring to the drawings, wherein like reference numbers correspond to like or similar components throughout the several figures, there is shown in FIGS. 1, 2, 3A, 3B, and 4, show an exemplary organ care system (OCS) is generally indicated at 100 for transport of a donor organ, which in the illustrative example described herein is a heart 200, or donor organ. The OCS 100, or system 100, includes an optical apparatus 70 for monitoring a condition of the donor heart 200 during transport of the donor heart 200 by the OCS 100. By monitoring the heart 200 during transport using the system, or organ care system 100, described herein including optical apparatus 70, organ care system (OCS) providers are enabled by the data and information generated by the optical apparatus 70 and system 100 to evaluate a suboptimal heart they otherwise wouldn't risk using, such that the system, method and apparatus for monitoring a donor organ, as described herein, is advantaged by potentially increasing the percentage of donor organs which are transplanted.

The organ care system (OCS) 100 shown in FIG. 1, in a non-limiting example, includes a basin 10 for receiving the donor organ 200, which may be a donor heart 200, a reservoir 20 for containing a normothermic fluid 30, such as normothermic blood, which is circulated via a perfusion circuit 40 by a pump 50 in fluid communication with the perfusion circuit 40. The system includes an oxygenator 60 for oxygenating the normothermic fluid 30 during circulation through the perfusion circuit 40 to perfuse the donor heart 200 with oxygenated blood.

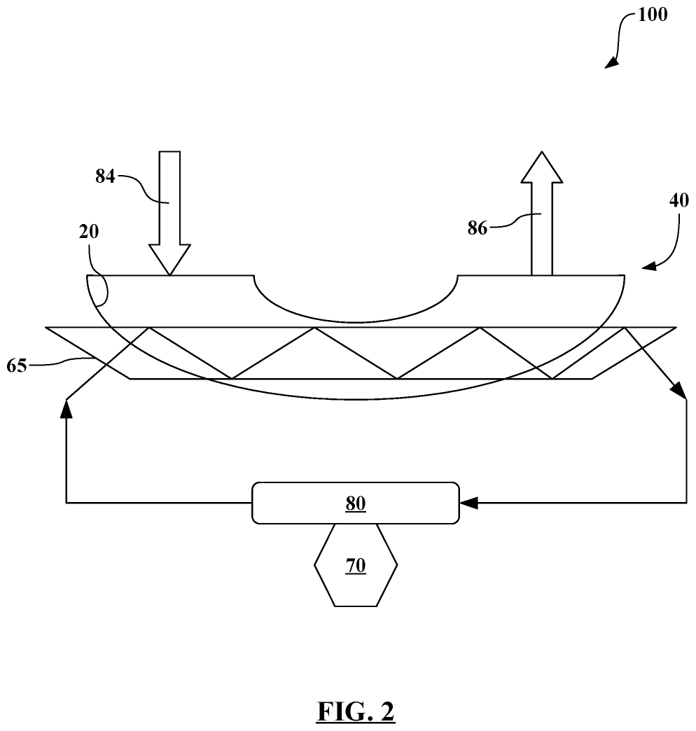

FIG. 2 shows a light bar 65, which operates via total internal reflection (TIR), which may alternatively be called an optical element 65. Additionally, FIG. 2 shows a normothermic blood inlet 84 and a normothermic blood outlet 86.

The system 100 integrates an optical apparatus 70 which is configured for continuous monitoring of and detecting trending of normothermic fluid 30 (for example, normothermic blood) for cardiac biomarkers or oxygenation levels, which enables real time monitoring of cardiac muscle health of the heart 200 during transport. System 100 includes an optical apparatus 70 that incorporates an optical configuration integrated with an electronics module that collects and transmits real-time cardiac monitoring data. The optical apparatus 70 can also be referred to herein as an optical configuration, an optical device, a monitoring device, and/or a continuous monitoring device.

An inframarker sensor/analyzer, where the inframarker being sensed and analyzed is indicative of a biomarker of a pathophysiological state, and/or a cryoprotective agent (CPA). Note that the inframarker sensor may be referred to as an infrasensor. Another example, without limitation, could be modulating the dosage of CPA in cases of CPA toxicity. Psychophysiological baseline may refer to several different baseline techniques available for completing psychophysiological research, as will be recognized by those having ordinary skill in the art.

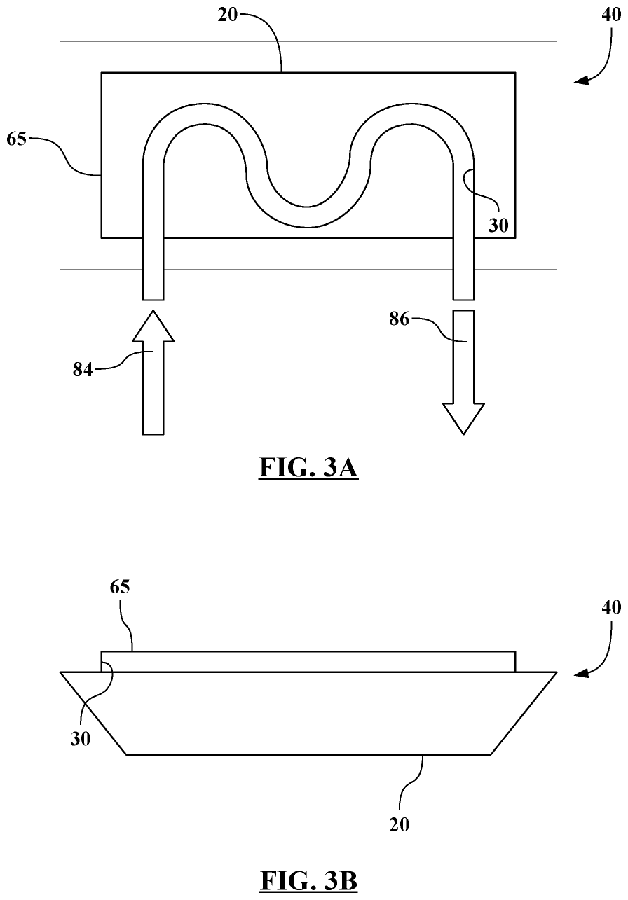

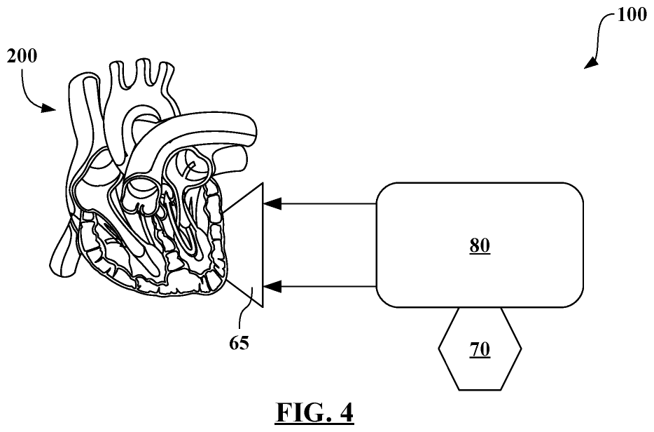

The optical configuration including the optical apparatus 70 can integrate with current heart transport solutions such as the OCS 100 shown in FIG. 1, for example, using one or more integration configurations, such as the example integration configurations shown in FIGS. 2-4. These could be in-line, flow-based solutions (examples A and B shown in FIGS. 2, 3A and 3B, respectively), and integrated solution (example C shown in FIG. 4) at multiple sites such as reservoirs 20, perfusion circuits 40, etc. The optical apparatus 70 can also be applied directly on the organ via an optical transmembrane. The optical apparatus 70 may include a controller 80, for controlling and/or operating the optical apparatus 70.

The optical apparatus 70, for example, via the controller 80, can be in wired or wireless communication with a database for collection of data generated by the optical apparatus 70 during continuous monitoring of the normothermic fluid 30 and/or the donor heart 200 for detection of biomarkers indicative of cardiac muscle health, including, without limitation, indicative of chilling injury/cryoinjury, and/or ischemic injury and/or indicative of CPA toxicity. Referring to FIGS. 2-4, additional illustrations, and descriptions of various exemplary configurations of the monitoring apparatus 70 are provided and incorporated by reference into this disclosure.

A current standard for donor heart preservation consists of cold organ storage with controlled temperature preservation. A donor heart subjected to cold storage can be susceptible to freezing injuries with protein denaturation, and transcriptomic changes dependent on the ischemic tolerance. Improvements with transport carriers for donor hearts involve perfusing a warm, beating donor heart with oxygenated blood as it is being transported to the operating room. The system and method described herein and illustrated by the accompanying figures provides the advantage of monitoring the condition of a donor heart during transport, providing data which can be used to evaluate the heart condition for transplant.

With further improvements in heart preservation, the system of continuous monitoring described herein can be extended to heart banks, potentially enabling long term storage for preserved hearts, similar to blood banks, where the data provided by the described monitoring system can be used to assess the condition of the preserved heart during the storage period. This provides the advantage of expanding the pool of available hearts between donors and recipients, by enabling extended transport times and/or organ storage for transplant.

Using data collected from the optical apparatus 70 during continuous monitoring of the heart 200, continuous trends of cardiac biomarkers from hearts 200, for example, pre-transplant with donor baselines, during transport and/or storage (for example, in a donor heart bank), and post-transplant with organ recipient baselines can be centrally monitored in a cloud based solution/on-premise datacenter with smart analytics that looks for changes to baseline in the transmembrane or trans-dermally derived inframarker levels. In one example, the system 100 and monitoring function is configured to trigger notifications to activate a therapeutic response (such as administering an antiarrhythmic medication, or automatic cardioversion) in reverting the heart back to normal. Therapeutic responses and dosage levels can be automated based on configuring the trigger notifications or alert clinical personnel to activate cardiovert protocol.

Further monitoring can be extended post heart transplant via transdermal optical based wearables, thereby enabling monitoring the baseline biomarkers in the recipient, for near term graft rejection, and/or monitoring for long term complications such as cardiac allograft vasculopathy. This may include, without limitation, running computational models on transdermal monitoring of pre-heart transplant patients and/or transmembrane monitoring of hearts stored in heart bank, to match recipient baseline levels for the donor heart pre-and-post heart transplant. This may further include, without limitation, predictive modelling toward lifetime expectancy and rejection rates enables optimal donor-recipient matching.

The example of monitoring a donor heart during transport and/or storage is non-limiting, and it would be anticipated within the scope of disclosure that the donor organ monitoring system described herein could be used with other donor organs. For example, and without limitation, transplantable organs including the liver, kidneys, and/or lungs.

Note that kidney dialysis may also be monitored, in addition to other systems with circulating fluids. Whether you do home hemodialysis or in-center hemodialysis, you'll rely on a hemodialysis machine to filter your blood. The machine filters your blood through a dialyzer, also known as an artificial kidney, with built-in safety checks to be sure the process is safe and effective. Dialysate is a fluid that is made up of water, electrolytes, and salts. During dialysis, dialysate helps to clean your blood inside the dialyzer by removing waste products and balancing electrolytes. Your nephrologist will prescribe the dialysate that is right for your body's needs.

Skilled artisans will recognize additional transplantable organs that may be monitored by the methods and systems described herein. Current standard-of-care does not include longitudinal monitoring of cardiac biomarkers for end-organ damage or multiple organ dysfunction syndrome, ECMO or dialysis patients. This is often done using Extracorporeal Membrane Oxygenation (ECMO) and/or sometimes along with dialysis. As will be recognized by skilled artisans, extracorporeal refers to occurring or based outside the living body.

ECMO is a therapy that adds oxygen to your loved one's blood and pumps it through their body like the heart. The process takes place outside the body. You may hear of the process called extracorporeal life support (ECLS). ECMO is like a heart-lung machine used in heart surgery and can be used for longer periods of time. ECMO temporarily takes over the work of the heart and lungs so they can rest and heal. ECMO is used when usual treatments are not working. ECMO does not cure heart or lung disease, it only provides time for the patient's heart or lungs to heal.

Veno-arterial extracorporeal membrane oxygenation (VA-ECMO) is an established strategy for cardiopulmonary support with increasing use in patients with cardiovascular collapse. In contrast to veno-venous ECMO, for which the primary focus is gas exchange, VA-ECMO allows for blood to be drained from a central vein and returned to the arterial system. This allows for both respiratory and circulatory support. The classic indication for VA-ECMO is cardiogenic shock, defined by decreased cardiac output and myocardial contractility resulting in tissue hypoperfusion. Myocardial infarction (MI), colloquially known as heart attack, is caused by decreased or complete cessation of blood flow to a portion of the myocardium.

There are more than 500,000 people living in the United States with end-stage renal disease (ESRD). The prevalence of coronary artery disease (CAD) in patients with dialysis contributes significantly to elevated mortality and CAD prevalence. Free radicals produced during reperfusion contribute to myocardial stunning, with the duration of ischemia determining the magnitude of free radical formation. With free radical formation, the generation of reactive oxygen species occurs, and they are then available to interact with lipids within cell membranes or with other cellular components such as calcium to promote myocardial stunning. These reactions activate calcium-dependent protease activity and consequently troponin I proteolysis.

Note that these techniques may be used on markers of organ failure. In heart transplant, cardiac biomarkers are relevant. In dialysis, acute kidney failure or acute kidney disease (AKD) as well as chronic kidney disease (CKD) and/or end stage renal disease (ESRD) need to be monitored. In lung transplant, biomarkers of acute lung injury are used. In liver transplant, biomarkers of liver injury, and inflammatory markers are all needed to monitor the underlying state of these organs.

With a continuous monitoring system, the mortality rate in patients can be directly informed by the biomarker trend. In patients dependent on long-term dialysis, the strain of dialysis is also reported to cause cardiac stunning. This adverse effect of dialysis on heart health can also be monitored by cardiac biomarker/inframarker trending. In post cardiotomy ECMO, commonly required after aortic arch repair, cardiac biomarker/inframarker trending could indicate outcome probability of irreversible myocardial damage. Further, biomarkers such as al-troponin can predict successful weaning from venoarterial ECMO. In cardiac surgery patients, ECMO is increasingly considered as a viable rescue strategy with an increased survival to discharge rate which can directly benefit from continuous biomarker monitoring.

Patient-controlled analgesia (PCA) is a type of pain management that lets you decide when you will get a dose of pain medicine. In some situations, PCA may be a better way of providing pain relief than calling for someone (typically a nurse) to give you pain medicine. With PCA you don't need to wait for a nurse.

Cardiotoxicity is heart damage that arises from certain cancer treatments or drugs. It can develop years after cancer treatment, especially in adults who received cancer treatment during childhood. Certain types of cancer treatments have a higher risk for cardiotoxicity. Note that some, without limitation, include deploying treatment options based on analysis of inframarkers in cardiac cases, ECMO, transplant organs, pain management, and cancer cases. Skilled artisans will recognize the terminology and mechanisms used to treat the different types of disease factors.

Dosage, or dose, modulation may refer to dosage compensation or inputting greater quantities of dosage at the beginning of the input. Skilled artisans will recognize further uses for the term dose modulation or dosage modulation.

The term “comprising,” and variations thereof, as used herein, is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide more specific embodiments and are also disclosed. As used in this disclosure and in the appended claims, the singular forms “a,” “an,” “the,” include plural referents unless the context clearly dictates otherwise.

The detailed description and the drawings or figures are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

Furthermore, the embodiments shown in the drawings, or the characteristics of various embodiments mentioned in the present description, are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

1. A method for monitoring a condition of a donor organ using an optical device, comprising:
providing a reservoir of normothermic fluid;
providing a perfusion circuit for the normothermic fluid;
providing a pump in fluid communication with the perfusion circuit;
providing an oxygenator for oxygenating the normothermic fluid during circulation through the perfusion circuit to perfuse the donor organ with oxygenated blood;
providing an optical device integrated within the reservoir, or the pump or the oxygenator, or in-line flow-based within the perfusion circuit through which the normothermic fluid flows; and
using total internal reflection for the optical device.

2. The method of claim 1, wherein the reservoir, pump, oxygenator, or in-line perfusion circuit of normothermic fluid and the optical device are configured to monitor a physiological baseline of the donor organ during transport.

3. The method of claim 1, wherein a donor organ's physiological baseline is established pre-transport via transmembrane measurements of a donor organ in an organ bank; the organ bank containing a donor organ selected from a group of a heart, a liver, a lung, or a kidney.

4. The method of claim 1, wherein a donor organ's physiological baseline is established pre-transport via transdermal measurements in a donor organ.

5. The method of claim 1,
using computational models on one of pre-transplant transdermal or transmembrane baselines to optimally match recipient baseline pre-and-post organ transplant.

6. The method of claim 1, using computational models on one of pre-transplant transdermal or transmembrane baselines to predict lifetime expectancy and rejection rates.

7. The method of claim 1, further comprising:
detecting levels of cryoprotective agents.

8. The method of claim 1, further comprising:
detection of biomarkers indicative of organ health.

9. The method of claim 1, further comprising:
using inframarker based analysis and dosage modulation in the donor organ; and
wherein levels of cryoprotective agents are analyzed in relation to organ health, and
wherein a therapeutic response is initiated, or the levels of cryoprotective agents are adjusted, in real-time to avoid organ toxicity.

10. A system for monitoring a condition of an organ, comprising:
a circulation system for blood, exterior to a body, but connected to the body; and
using the circulation system for adding filtration for the blood, and wherein the filtered blood goes back into the body.

11. The system for monitoring a condition of an organ of claim 10, further comprising:
an external circulation system integrates an optical device to monitor a physiological baseline of the organ.

12. The system for monitoring a condition of an organ of claim 11, wherein the system is configured to monitor for dialysis-induced myocardial stunning from repetitive ischemic injury.

13. The system for monitoring a condition of an organ of claim 12, wherein the system is configured to monitor and analyze reverse remodeling of the organ heart in patients on heart failure medication.

14. The system for monitoring a condition of an organ of claim 13, further comprising:
external circulation system integrates an optical device to monitor the physiological baseline of the organ.

15. A system for monitoring a condition of an organ of claim 14, wherein the system is configured to monitor adverse effects of chronic kidney disease or end stage renal disease on organ health.

16. A system for monitoring a condition of an organ of claim 15, wherein the system is configured to monitor dialysis patients for the adverse effects of medications on organ health.

17. A method for monitoring a condition of a heart or lung, comprising:
using extracorporeal membrane oxygenation (ECMO) to monitor oxygenation levels in circulation; and
integrating an optical device to monitor a physiological baseline of the heart or lung.

18. The method for monitoring a condition of a heart or lung of claim 17, further comprising:
using total internal reflection for the optical device.

19. The method for monitoring a condition of a heart or lung of claim 18, wherein the method is configured to monitor for cardiotoxic medication overdose.

20. The method for monitoring a condition of a heart or lung of claim 18, wherein the method is one of:
configured to monitor for perioperative myocardial infarctions, or
configured to monitor for organ rejection during organ transplant.
